(19) United States
(12) Patent Application Publication
(10) Pub. No.: US 2017/0216058 A1
(43) Pub. Date: Aug. 3, 2017

(54) PROSTHETIC SLEEVE

(71) Applicant: Nottingham Trent University, Nottingham (GB)

(72) Inventors: Tilak Dias, Nottingham (GB); Colin Cork, Norfolk (GB); Jose Carlos Oliveira, Nottingham (GB); Richard Arm, Nottingham (GB); William Hurley, Nottingham (GB)

(21) Appl. No.: 15/502,420

(22) PCT Filed: Jul. 31, 2015
(86) PCT No.: PCT/GB2015/052213

§ 371 Date: Feb. 7, 2017

(30) Foreign Application Priority Data
Aug. 8, 2014 (GB) 1414128.7

(51) Int. Cl. A61F 2/78

(52) U.S. Cl. CPC A61F 2/7812; A61F 2002/7837; A61F 2002/7887

(57) ABSTRACT

A sleeve for mounting a prosthetic unit on a limb is formed in a knitted fabric comprising elastic yarns to allow circumferential extension of the sleeve to grip the limb. Adherent fibres are integrated within the fabric and exposed on the internal surface of the sleeve to provide additional grip. Such fibres can be silicone or silicone based yarns. An end of the sleeve is adapted to couple with a prosthetic unit. The knitted fabric may include yarns of restricted elasticity to limit longitudinal extension of the sleeve, and the elastic yarns and the yarns of restricted elasticity are typically located in discrete sections (2, 4, 6) of the sleeve. Such discrete sections will normally extend longitudinally in the sleeve. The knitted yarns at the end of the sleeve are normally bonded directly to a coupling element (8, 10, 12) for attachment to a prosthetic unit.

This invention relates to artificial limbs, and particularly to the attachment of prosthetic units to limbs. Attachment devices for prosthetics are normally based on a sleeve adapted to receive the stump of the limb, at the end of which a unit is fitted for coupling to the respective prosthetic. Such arrangements are disclosed in U.S. Pat. No. 6,231,617; US Patent publication Nos. 2005/0240283 and 2002/0183859; and International Patent publication Nos. WO 00/51531 and WO 00/51537. The disclosures of all of these documents are hereby incorporated by reference.

Prosthetic legs are fitted to the stump of an amputee using a flexible sleeve. Current prosthetic sleeves are constructed from textile fabrics coated with an impermeable sheet of silicone rubber. Consequently, sweat is not transported away from the skin and accumulates within the sleeves and has then to be drained. The present invention is directed at a breathable prosthetic sleeve constructed from textile fibres alone. Textile materials can be tailored to particular needs as there are a wide range of fibres, yarn types and structures that can be employed.

To be comfortable, a sleeve for use in attaching a prosthetic unit to a limb should not only facilitate the movement of moisture from the skin, but movement of the sleeve relative to the limb must be minimized without applying undue pressure on the flesh against which it is held. As far as possible, the limb must also be exposed to air. The present invention seeks to address these issues and provide a sleeve which meets these objectives.

According to the invention, a sleeve for mounting a prosthetic unit on a limb is formed in a knitted fabric comprising elastic yarns allowing circumferential extension of the sleeve to grip a said limb; and adherent fibres integrated within the fabric and exposed on the internal surface of the sleeve to provide additional grip. The adherent fibres are normally silicone or silicone based, and preferably part of the knitted structure of the fabric. They may be disposed in panels formed on the internal surface of the sleeve to provide said additional grip. Such panels can be integrated sections of the sleeve knitted with yarns having adherent characteristics.

A sleeve according to the invention may include yarns of restricted elasticity to limit longitudinal extension of the sleeve. Such yarns will normally be part of the knitted fabric, and the elastic yarns and the yarns of restricted elasticity can be located in discrete sections of the sleeve. Such discrete sections will typically extend longitudinally in the sleeve.

In a particular embodiment a sleeve according to the invention has first and second integrated elongate sections knitted with yarns having different characteristics. The first sections comprise yarns of restricted elasticity to limit longitudinal extension of the sleeve, and the second sections comprise elastic yarns to allow circumferential extension of the sleeve to grip the limb. An end of the sleeve is adapted to couple with a prosthetic unit. Regions of the internal surface of the sleeve are adapted to provide additional grip, by the inclusion of individual fibres having adherent characteristics in said regions.

The end of a sleeve according to the invention may be integral with a plastics moulding bearing a coupling for a prosthetic unit. Alternatively, the end of the sleeve may take the form of a plastics moulding adapted to receive a prosthetic unit. In either case, at least one of the moulding and coupling can be shaped to match the stump of the limb upon which the sleeve is to be mounted, and can be extended over the end of the sleeve. Such an extended moulding can be formed with longitudinal slots to preserve the permeability of the sleeve, and may be attached to the sleeve to restrict the longitudinal extensibility of the sleeve. These elements; the moulding and coupling, can be manufactured using 3-D printing. Their shape can be established from point cloud data generated by three-dimensional scanning of the stump of the limb. Whatever form it takes, and however it is manufactured and assembled, some cushioning can be provided, and the end of the sleeve adapted to cover the stump can allow the passage of air therethrough for ventilation.

In order to provide uniform engagement with the limb, in a sleeve according to the invention the first and second knitted sections will normally extend along the or the substantial length of the sleeve and be spaced around the sleeve circumference, normally arranged in a repetitive sequence. Where the regions providing the additional grip are in the form of panels, these panels may form third integrated sections that also extend along the or the substantial length of the sleeve. Some or all of the sections may be tapered or otherwise three-dimensionally (3D) shaped to adapt the shape of the sleeve as appropriate to conform with the shape of the limb upon which the sleeve is to be mounted. Generally the sleeve will be shaped and/or have a degree of taper for this purpose. The yarns the second sections are typically elastomeric yarns. The yarns of the first sections are typically one of Zylon (p-phenylene-2,6-benzobisoxazole), para-aramid, high modulus polyester, and polyamide yarns. The circumferential extensibility of the sleeve can vary along its length, and such varying extensibility may be determined by variations in the circumferential width of the second sections along their length. Such varying extensibility may also be selected according to characteristics of the limb upon which it is to be mounted, and those characteristics can be established from point cloud data generated by three-dimensional scanning of the limb. This enables the sleeve to be engineered to provide graduated compression to encourage venous flow in the limb upon which it is to be mounted.

The invention will now be described by way of example and with reference to the accompanying schematic drawings, wherein:

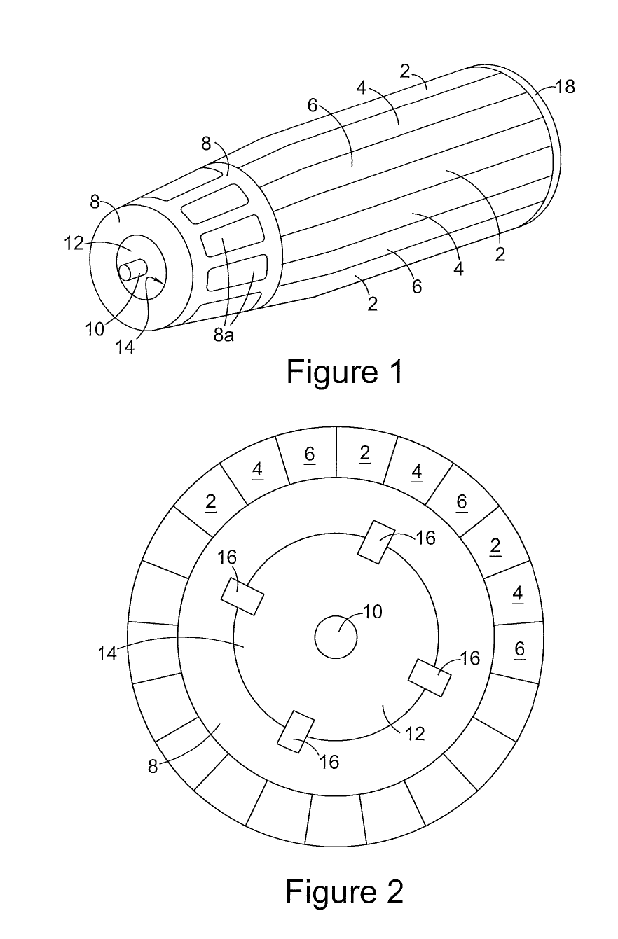

FIG. 1 is a perspective view of a sleeve according to the invention;

FIG. 2 is an end view of the sleeve of FIG. 1 showing the coupling of a prosthetic unit thereto;

FIG. 3 is a cross-section of the end of a sleeve according to the invention showing details of a cushioning assembly;

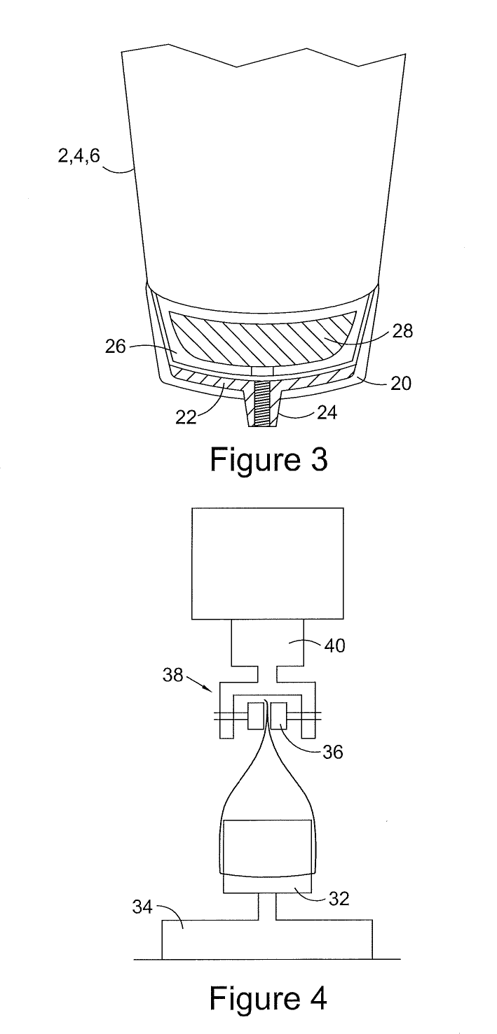

FIG. 4 shows a machine for testing the gripping force achieved and pressure applied by a sleeve according to the invention mounted on a boss in the machine;

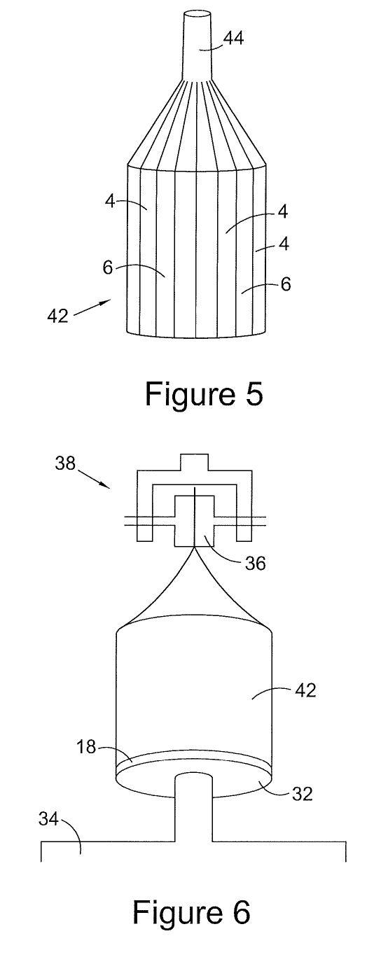

FIG. 5 shows the sleeve prepared for testing on the machine of FIG. 4;

FIG. 6 shows the sleeve of FIG. 5 mounted on the boss of the machine for testing;

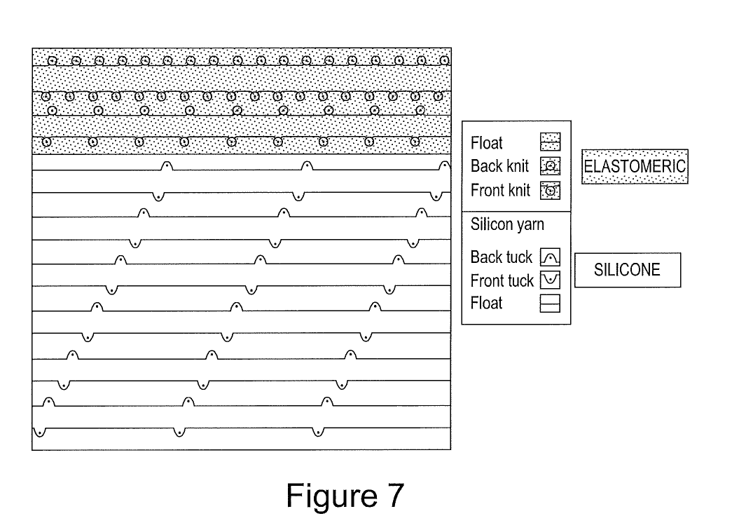

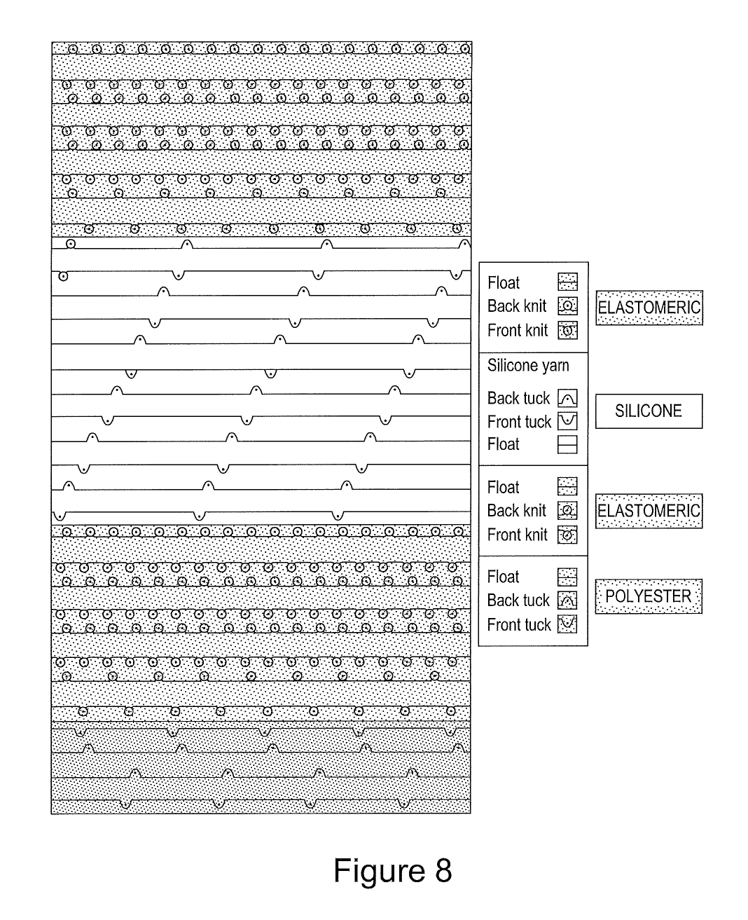

FIGS. 7 and 8 show alternative knitting patterns for sleeves according to the invention;

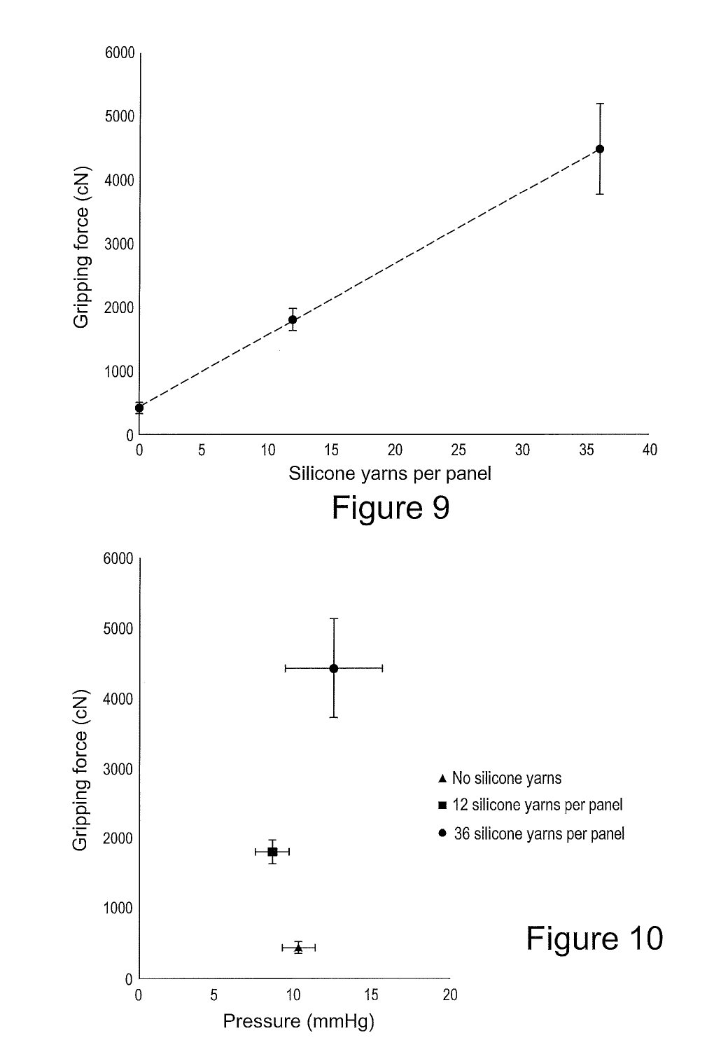

FIGS. 9 and 10 are graphs showing gripping forces and pressures established on the boss by different sleeves of the invention; and

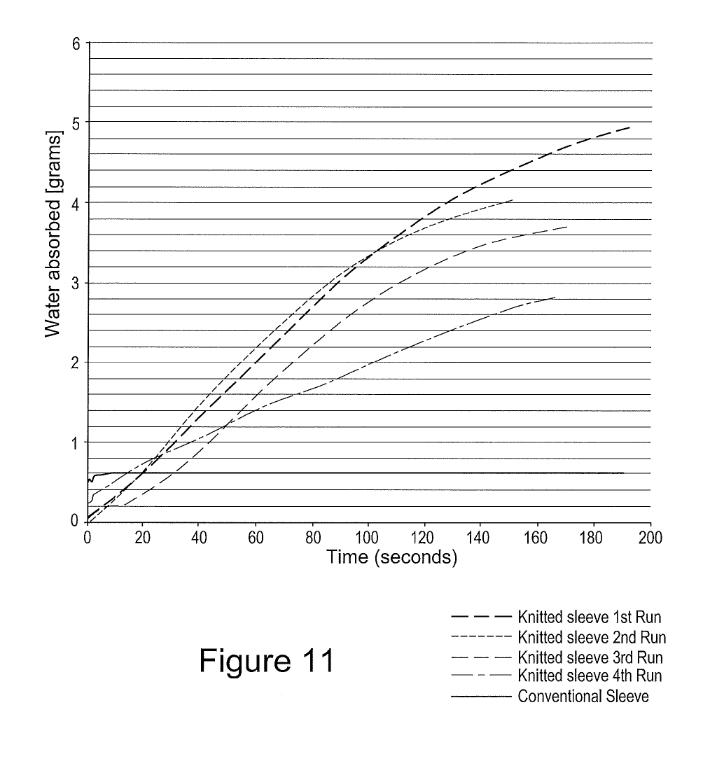

FIG. 11 is a graph illustrating the water absorbency of a sleeve of the invention.

The sleeve of FIG. 1 is in the form of a knitted fabric tube which is three dimensionally shaped towards one end at which a prosthetic unit (not shown) may be attached. The shape is typically cylindrical over most of its length from the other, open, end, but the exact profile may be selected for the particular limb upon which it is to be mounted. The fabric defines separate elongate sections 2, 4 and 6 arranged in a repetitive sequence around the tube circumference. The first sections 2 are knitted with relatively non-extensible high modulus yarns, such as Zylon, aramid, polyester or polyamide yarns, to restrict the longitudinal extension of the sleeve. These sections may also be knitted as plain, rib, interlock or purl structures to stabilize the tubular assembly. The second sections 4 are knitted with double covered elastomeric yarns to enable circumferential stretching and allow the sleeve to grip the limb upon which it is mounted. The use of elastomeric yarns facilitates the introduction of a graduated compression characteristic matched to the limb upon which it is to be mounted, and to encourage the venous flow in the limb. The third sections 6, which may be discontinuous to form spaced panels, are knitted with yarns such as silicone yarns, having adherent characteristics to prevent slippage of the sleeve when fitted. Suitable silicone coated nylon yarns are available from Massebeuf Textiles Sas of Pont de Labeaume, France. Different panel sequences may be adopted and in a preferred arrangement described below, the repeated sequence is of four sections; two of elastomeric yarns, one of silicone coated yarns, and one of relatively non-extensible yarns. In another variant, the first sections can be omitted such that the sleeve is knitted only with elastomeric yarns and adherent yarns. In some embodiments, the third sections may not be required and as required, yarns or fibres having adherent characteristics can be incorporated in the first or second sections to provide additional grip.

The second sections 4 enable the sleeve to be extended circumferentially to fit over and grip the limb requiring a prosthetic unit. Their elastic extensibility will be determined by the yarns used; the knitted structure, the tightness of the knitting, and the yarn dimensions. These can be selected to match the dimensions and other characteristics of the limb, and can vary along the length of the sleeve. Typically, these sections will be three dimensionally shaped as shown towards the one end. As noted above, the dimensions of the limb can be determined by three-dimensional scanning, and the dimensions and characteristics established using point cloud data generated by such scanning.

The smaller end of the sleeve of FIG. 1 is closed by a moulded cap 8, the interior of which can be shaped to match the stump of the limb upon which the sleeve is to be mounted. It will also normally include a cushion (not shown) for engagement with the stump. A bolt 10 is fixed in the cap 8 and protrudes to be coupled to a prosthetic unit (not shown). The entire cap 8 can be permanently attached to the fabric tube such that the sleeve and cap with the protruding bolt form a single integral body, but in some embodiments the bolt 10 is fixed in a base 12 which is itself received in a recess 14 in the cap 8. This arrangement is illustrated in FIG. 2. The base 12 and recess 14 can be complementarily conical to ensure a secure support for the bolt and prosthesis coupled thereto, with the base held in place by locking clips 16. The cap 8, whether in unitary or modular form, can be moulded in a porous material allowing the passage of air to provide ventilation for the stump when the sleeve is fitted to a limb. The cap will normally cover the end of the sleeve as shown in FIG. 3, but can be extended longitudinally as shown in FIG. 1 and, if attached to the sleeve, serve to restrict longitudinal extension of the sleeve at its end. This can be beneficial particularly in the variant referred to above in which the yarns of restricted elasticity (those of the first sections 2) are omitted. The extended portion is formed with slots or openings 8A for ventilation. A particular sleeve end with no such extension is described below with reference to FIG. 3.

The other, larger end of the sleeve is formed with a band 18. This is loosely knitted so as not to alter the compressive characteristics of the sleeve, but facilitate its fitting and removal.

In the sleeve end shown in FIG. 3 the coupling element comprises a flexible silicone based membrane 20 containing a nylon umbrella 22 including a threaded boss 24 for receiving the complementary bolt of a prosthetic unit, and a silicone based spacer 26 supporting a cushion 28. The knitted yarns of the sleeve sections 2, 4 and 6 are bonded directly to the membrane 20, a portion of which extends over the cushion 28.

It will be appreciated that a range of materials may be used in sleeves of the invention to match the characteristics required, and that the nature and design of the cap will of course be selected according to the nature of the limb to which the sleeve is to be fitted. Particularly, the bolts shown in FIGS. 1 and 2 merely as examples of simple couplings. Depending upon the specific prosthetic unit to be installed, different coupling mechanisms can of course be used.

A prosthetic sleeve tester was designed in the form of a 12.0 cm diameter boss 10.0 cms long which was mounted in a tensile tester illustrated in FIG. 4. Suitable testers are available from Zwick Testing Machines Ltd of Leominster, United Kingdom. The boss 32 is mounted on a plinth 34 below a pair of jaws 36 in a clamp 38 attached to a piston 40. Operation of the machine withdraws the piston upward to separate it from the plinth 34 (and a boss 32 mounted thereon) at a chosen rate and monitors the resistance thereto provided by a specimen held therebetween.

Two sleeves were prepared specifically for testing on the designed machine. The first is shown in FIG. 5. It has a specimen cylindrical section 42 of 10.5 cms diameter to be mounted on the boss 32 as shown in FIG. 6 connected by a tapering section to a tab section 44 of 3 cms diameter to be held between the jaws 36 of the tester. The cylindrical section of each sleeve has no polyester (relatively inelastic) yarns, but comprises elastomeric yarns and silicone coated yarns arranged in circumferentially alternate elongate panels 4 and 6 extending substantially the length of the sleeve. The knitting pattern for the sleeve having four elastomeric yarns per panel (4) and twelve silicone per panel (6) is shown in FIG. 7. The second sleeve has a similar pattern, but with thirty-six silicone coated yarns per panel (6) rather than twelve. An alternative knitting pattern for a sleeve according to the invention is shown in FIG. 8. This has a circumferentially repeating sequence of four panels with four polyester yarns in the first eight elastomeric yarns in the second twelve silicone yarns in the third and eight elastomeric yarns in the fourth. The count of the polyester yarns is 167 dTex; the count of the elastomeric yarns is 800 dTex; and the count of the silicone coated yarns is 350 dTex.

Both a wooden and a metal (aluminium) boss were made and used, with most tests being undertaken on the wooden tester. However, it was realised that in the long-term, a metal tester might be more appropriate. A wooden boss provides a better match for the roughness of the human skin but could become polished with time. A metal boss would be more resistant to polishing and importantly, could be cleaned with an alcohol wipe between tests. A range of sleeves were produced for testing (FIG. 5). These were shaped with a 3.0 cm tab at the top so that they could be gripped within the jaws of the tester.

Tests were conducted on the two prepared sleeves, and on a reference sleeve of the same diameter knitted only with elastomeric yarns. The sleeves were mounted carefully on the cylindrical tester as shown in FIG. 6. A Surface Pressure Measurement Analyser (available from AMI Techno Co Ltd of Tokyo, Japan) was inserted between the sleeve and the boss in order to determine the applied pressures. The aim was to provide the required grip while keeping the applied pressures well below blood pressure (80-90 mmHg). It is important that pressures are kept as low as possible to provide comfort and to ensure that pressures never approach levels where tissue damage can occur. The tester was set to remove sleeves at a speed of 200 mm/min. The maximum force was then determined. The results were then plotted against the applied pressure and the results are shown in FIGS. 9 and 10. As can be seen, while each sleeve applied a substantially similar range of pressure to the boss 32, the gripping force is substantially increased by the inclusion of silicone coated yarns.

Tests were also conducted to examine the moisture absorption capability of sleeves of the invention against a conventional sleeve with an impermeable coating, mounted on the boss. For this purpose, a MK Gats system model M/K241 moisture absorbency tester, available from MK Sysems Inc of Peabody, Mass., United States of America, was used. This measures the mass of water absorbed per unit sample weight per unit time. The results for a knitted sleeve of the invention comprising just elastomeric yarns and adherent yarns relative to a known silicone lined sleeve are shown in FIG. 9. In the case of the conventional commercial sleeve (line A) some moisture travels down the interface between the boss and the sleeve, but no water is absorbed due to the continuous sheet of silicone covering the textile substrate. However, there is significant transport of moisture into the knitted sleeve of the invention. Line B shows the absorbency in the first test on the sleeve; lines B, C, and D the absorbency on the second third and fourth test on the same sleeve. This indicates that perspiration would be transported away from the skin using a sleeve of the invention, with a resultant improvement in skin health.

1. A sleeve for mounting a prosthetic unit on a limb, having an end adapted to couple with a said prosthetic unit, which sleeve is formed in a knitted fabric comprising elastic yarns allowing circumferential extensibility of the sleeve to grip a said limb; and adherent fibres integrated within and part of the knitted structure of the fabric and exposed on the internal surface of the sleeve to provide additional grip.

2. (canceled)

3. A sleeve according to claim 1 wherein the adherent fibres comprise at least one of silicone yarns, silicone coated polyamide yarns, and silicone coated polyester yarns.

4. A sleeve according to claim 1 wherein the adherent fibres are disposed in panels formed on the internal surface of the sleeve to provide said additional grip.

5. A sleeve according to claim 4 wherein the panels are integrated sections of the sleeve knitted with yarns having adherent characteristics.

6. A sleeve according to claim 1 wherein the knitted fabric includes yarns of restricted elasticity to limit longitudinal extension of the sleeve.

7. A sleeve according to claim 6 wherein the elastic yarns and the yams of restricted elasticity are located in discrete sections of the sleeve.

8. A sleeve according to claim 7 wherein the discrete sections extend longitudinally in the sleeve.

9. A sleeve according to claim 8 wherein the sections are arranged in a repetitive sequence around the sleeve circumference.

10. A sleeve according to claim 7 wherein the sections extend along the length of the sleeve and are spaced around the sleeve circumference.

11. A sleeve according to claim 1 wherein the elastic yams are elastomeric yarns.

12. A sleeve according to claim 6 wherein the yarns of restricted elasticity are one of polyester, polyamide, Zylon and aramid yarns.

13. (canceled)

14. A sleeve according to claim 1 wherein the end of the sleeve is integral with a plastics moulding adapted to receive a prosthetic unit.

15. A sleeve according to claim 14 wherein the moulding allows the passage of air therethrough.

16. A sleeve according to claim 14 wherein the moulding is shaped to match the stump of the limb upon which the sleeve is to be mounted.

17. A sleeve according to claim 16 wherein at least one of the moulding and coupling are manufactured using 3-D printing.

18. A sleeve according to claim 16 wherein the shape of the moulding is established from point cloud data generated by three-dimensional scanning of the stump of the limb.

19. A sleeve according to claim 1 wherein the circumferential extensibility of the sleeve varies along the length of the sleeve.

20. A sleeve according to claim 19 wherein the circumferential extensibility of the sleeve reduces towards the end adapted to couple with a said prosthetic unit.

21. A sleeve according to claim 19 wherein the elastic yarns are located in discrete longitudinal sections of the sleeve and wherein said varying extensibility is determined by variations in the circumferential width of said sections along their length.

22. A sleeve according to claim 16 wherein said varying extensibility is selected according to characteristics of the limb upon which it is to be mounted.

23. A sleeve according to claim 22 wherein said characteristics are established from point cloud data generated by three-dimensional scanning of the limb.

24. A sleeve for mounting a prosthetic unit on a limb, having an end adapted to couple with a said prosthetic unit, which sleeve is formed in a knitted fabric comprising elastic yarns allowing circumferential extensibility of the sleeve to grip a said limb; yarns of restricted elasticity to limit longitudinal extension of the sleeve; and adherent fibres integrated within and part of the knitted structure of the fabric and exposed on the internal surface of the sleeve to provide additional grip.

25. A sleeve according to claim 24 wherein the elastic yarns and the yarns of restricted elasticity are located in discrete sections of the sleeve.

26. A sleeve according to claim 25 wherein the discrete sections extend longitudinally in the sleeve.

27. A sleeve according to claim 26 wherein the sections are arranged in a repetitive sequence around the sleeve circumference.

28. A sleeve according to claim 24 wherein the circumferential extensibility of the sleeve varies along the length of the sleeve.

29. A sleeve according to claim 28 wherein the circumferential extensibility of the sleeve reduces towards the end adapted to couple with a said prosthetic unit.

30. A sleeve according to claim 28 wherein the elastic yarns are located in discrete longitudinal sections of the sleeve and wherein said varying extensibility is determined by variations in the circumferential width of said sections along their length.
